(19) United States
(12) Patent Application Publication
(10) Pub. No.: US 2024/0002926 A1
(43) Pub. Date: Jan. 4, 2024

(54) METHOD FOR IDENTIFYING AN INFECTIOUS AGENTS

(71) Applicants: INSTITUT NATIONAL DE LA SANTÉ ET DE LA RECHERCHE MÉDICALE, Paris (FR); UNIVERSITE PARIS EST CRETEIL VAL DE MARNE, Créteil (FR); ASSISTANCE PUBLIQUE HOPITAUX DE PARIS, Paris (FR)

(72) Inventors: Christophe RODRIGUEZ, Créteil (FR); Jean-Michel PAWLOTSKY, Créteil (FR); Vanessa DEMONTANT, Créteil (FR)

(21) Appl. No.: 18/254,399

(22) PCT Filed: Nov. 26, 2020
(86) PCT No.: PCT/IB2020/000983

§ 371 Date: May 25, 2023

(51) Int. Cl. C12Q 1/6869; C12Q 1/6806; G16B 30/10; G16H 50/20

(52) U.S. Cl. CPC C12Q 1/6869; C12Q 1/6806; G16B 30/10; G16H 50/20

(57) ABSTRACT

The present invention provides methods for detecting, identifying, classifying, quantifying and/or characterizing an infections agent. The invention relates to a method for detecting, identifying, classifying, quantifying and/or genetically characterizing an infections agent comprising the steps of: —providing a sample of nucleic acid sequences; —isolating high-quality nucleic acid sequences out of the sample of nucleic acid sequences; —isolating at least one non-human high quality nucleic acid sequence out of the high-quality nucleic acid sequences; —identifying a closest known sequence out of a plurality of known sequences, wherein the closest known sequence shares the highest amount of similarities with the at least one non-human high quality nucleic acid sequence among the plurality of known sequences, and wherein the plurality of known sequences comprises sequences of infectious agents.

### FIELD

The present invention relates to the field of medicine, in particular microbiology and infectious diseases.

### BACKGROUND

The direct detection, identification, classification, quantification and characterization of infectious agents is traditionally performed by means of methods based on culture, antigen detection/quantification, DNA or RNA genome detection/quantification by means of target amplification methods (qPCR, qTMA, LAMP, etc), and/or DNA or RNA sequence analysis by means of targeted sequencing (including Sanger sequencing or next-generation sequencing [NGS]). However, all of these approaches have limitations, listed in Table 1.

TABLE 1
Comparison of the abilities to identify infectious agents (pathogens) of microbiological
technologies used for their direct detection in routine laboratory testing.

| | | | DNA or RNA | Sanger-based | NGS-based | Shotgun |
|---|---|---|---|---|---|---|
| | | Antigen | detection/quantification | targeted | Targeted | Metagenomics |
| Pathogen | Culture | detection/quantification | by target amplification | Metagenomics | Metagenomics | MetaMIC |
| Bacteria | Partial | Targeted | Targeted | 16S | 16S | Yes |
| | (culturable) | | | | | |
| Fungi | Partial | Targeted | Targeted | ITS | ITS | Yes |
| | (culturable) | | | | | |
| Viruses | No longer used | Targeted | Targeted | No | No | Yes |
| Parasites | Partial | Targeted | Targeted | 18S/28S | 18S/28S | Yes |
| | (culturable) | | | | | |
| Plurimicrobial | Partial | No | Targeted | No | Yes | Yes |
| | (culturable) | | | | | |
| New | Limited | No | No | Limited | Limited | Yes |
| pathogen | | | | | | |
| Resistance | Yes | Targeted | Targeted | No | No | Yes (genotyping) |
| | (phenotyping) | | | | | |

ITS: Internal Transcribed Spacer; Culturable: means that only living agents that can grow in culture can be detected).

The symptoms of infectious syndromes are generally not specific for a viral, fungal, bacterial or parasitic etiology. However, medical microbiology has been artificially split into different subspecialties corresponding to each family of pathogens, principally because the techniques to diagnose these infectious agents were different.

The main limitation of state-of-the-art microbiology technologies is the limited spectrum of infectious agents detected. Indeed, except bacterial/fungal cultures that grow without a priori, these methods can detect only a very limited number of predefined infectious agents (one to less than 20 pathogens, including bacteria, viruses, fungi and/or parasites, for current syndromic qPCR panels for example). The list of predefined agents that can be detected and characterized is based on the frequency of these pathogens as causal agents in the corresponding infectious syndromes, as described in epidemiological studies. However, many infectious agents that can be responsible for these infections are ignored, while their frequencies constantly vary and may dramatically increase in the context climate changes, massive migrations, pandemics, new medical practices (e.g. transplantation, immune suppression, antiinfectious therapy, . . . ), etc. These changes cannot be reflected by current diagnostic assays searching for limited panels of predefined agents that would need to be constantly updated and increased, generating high costs for development and accreditation in customer laboratories.

This situation emphasizes the need for a technology capable to detect, identify, classify, quantify and characterize without a priori any pathogen(s) responsible for a human or animal infection. In addition, there is a need for a technology with the ability to discover new, thus far unknown infectious pathogens in routine practice, in order to fill epidemiological and pathophysiological knowledge gaps.

There is also a need for a technology capable to provide information about the amount of any pathogen present in an analyzed sample. In particular situations, this information is necessary to measure the severity of the disease, confirm the role of the pathogen in the symptoms, establish a prognosis of the infection, make a therapeutic decision and/or follow the efficacy of anti-infectious treatments. Currently, quantification is possible only with target amplification methods and for a small number of predefined pathogens on a single-pathogen assay basis (e.g. HIV, CMV, HCV, HBV). For other infectious agents, only in-house single-pathogen assays have been developed that cannot be transferred to routine diagnostic laboratories and the markets are too small to guarantee the development of standardized commercial assays in the future.

Standard cultures can diagnose bacterial or fungal infections, but they are time-consuming and the identification can be flawed by the performance of the culture, the need for the pathogen to be alive, the characteristics of pathogens that do not grow well in culture or require specific conditions, and/or by the administration of antibiotics. Recent targeted metagenomics tools provided an alternative to culture. However, their performance proved to be inferior to that of classical culture.

Thus, there is a definite need for a new fast and reliable method for pathogen identification without an a priori.

### SUMMARY OF THE INVENTION

The present invention provides methods for detecting, identifying, classifying, quantifying and/or genetically characterizing an infectious agent. The present invention fulfills the needs identified above. In particular, the present invention is defined by the claims.

### DETAILED DESCRIPTION

The present invention relates to a method for identifying an infectious agent comprising the steps of:
a. providing a sample of nucleic acid sequences;
b. isolating high-quality nucleic acid sequences out of the sample of nucleic acid sequences;
c. isolating at least one non-animal high-quality nucleic acid sequence out of the high-quality nucleic acid sequences;
d. identifying a closest known sequence out of a plurality of known sequences, wherein the closest known sequence shares the highest amount of information with the at least one non-animal high-quality nucleic acid sequence among the plurality of known sequences, and wherein the plurality of known sequences comprises sequences of infectious agents, preferably at least one fungal discriminant gene of interest, and wherein said identification indicates the infectious agent.

This method makes it possible to detect, identify, classify, quantify and/or genetically characterize an infectious agent.

As used herein, the term “infectious agent” refers to a microorganism that causes an infection in an animal. Usually, the organisms are viruses, bacteria, parasites, protozoa and/or fungi.

As used herein, the term “animal” denotes all mammalian animals including humans. It also includes an individual animal in all stages of development, including embryonic and fetal stages. The term encompasses farm animals (pigs, goats, sheep, cows, horses, rabbits and the like), rodents (such as mice), and domestic pets (for example, cats and dogs). The method of the present invention is particularly suitable for identifying an infectious agent in a human.

Unlike other known techniques, the identification of the infectious agent can be performed accurately enough to be applied to the diagnosis of infections and their causal infectious agent(s). Specifically, the method can discriminate infectious agents of interest from contaminants. To do so, the method can further comprise a step consisting in isolating high-quality nucleic acid sequences out of a sample deprived of any nucleic acid sequence of interest. All of the identified sequences are thus considered to be contaminants and disregarded if found among the sample of nucleic acid sequences.

The method can also comprise a further step consisting in repeating steps a) to d) on a sample of nucleic acid sequences containing at least one known sequence. This step allows for validating the conditions of use of the method and for detecting any anomaly. In addition, the number of sequences belonging to one infectious agent correctly identified can be interpreted using a cut-off to determine whether the sample should be considered negative or positive for the presence of the infectious agent. The presence of the infectious agent (positive detection) can be listed in a report usable for medical interpretation in a microbiology laboratory.

The method can also comprise any of the following steps: quantifying the load of the pathogen, reconstructing the infectious agent's genome and making variant calling to identify nucleotide or amino acid differences as compared to a reference sequence.

As used herein, the term “nucleic acid sequence” refers to a DNA or RNA molecule in single- or double-stranded form. An “isolated nucleic acid sequence” refers to a nucleic acid sequence which is no longer in the natural environment from which it was isolated, e.g. the nucleic acid sequence in a cell.

The sample of nucleic acid sequences may thus consist of a bulk of DNA and RNA sequences, but RNA sequences may be sufficient to achieve the goals of the method of the present invention. The sample can be obtained in any way. The nature of the samples that can be collected from patients or animals is very diverse. Indeed, the technique has been validated on tissues (frozen and paraffin-embedded biopsies from various organs) and body fluids (cerebrospinal fluid, bronchoalveolar lavage, sputum, whole blood, plasma, serum, pus, urine, aqueous humor, bone marrow, ascites, etc).

Preferably, a management tool can monitor a plurality of samples from a plurality of patients or animals and allow for tracking the sample of interest anonymously.

In some embodiments, step a) comprises a substep consisting in extracting the nucleic acid sequences, and wherein said substep is monitored so as to generate information comprising at least the progress of extraction and the origin of the sample.

In order to provide the sample of nucleic acid sequences, pre-extraction consisting in a combination of mechanical, enzymatical and chemical lysis of the sample and extraction consisting in purification of nucleic acids by removing membranes, lipids, proteins and any other cell or extracellular component to provide high quality nucleic acids can be performed.

The method of the present invention is particularly efficient for identifying an infectious agent exclusively from RNA sequences. An environmental control (negative control) and a positive control (containing 8 bacteria, 2 fungi and 4 viruses) can be included according to recommendations of the ISO 15189 norms.

In some embodiments, a library of nucleic acid sequences of the extract is prepared and sequencing said nucleic acid sequences is then performed.

As used herein, the term “sequencing” means a process for determining the order of nucleotides in a nucleic acid. A variety of methods for sequencing nucleic acids is well known in the art and can be used. In some embodiments, next-generation sequencing is carried out. As used herein, the term “next-generation sequencing” has its general meaning in the art and refers to sequencing technologies having increased throughput as compared to traditional Sanger- and capillary electrophoresis-based approaches, for example with the ability to generate hundreds of thousands or millions of relatively short sequence reads at a time. Next-generation sequencers are well known in the art and can include a number of different sequencers based on different technologies, such as Illumina (Solexa) sequencing, Roche 454 sequencing, Ion torrent sequencing, SOLiD sequencing, PacBio sequencing, and the like. An example of a sequencing technology that can be used in the present methods is the Illumina platform. The Illumina platform is based on amplification of DNA (after reverse transcription for RNA) on a solid surface (e.g., flow cell) using fold-back PCR and anchored primers (e.g., capture oligonucleotides). For sequencing with the Illumina platform, DNA is thus fragmented, and adapters are added to both terminal ends of the fragments (see the preceding step). DNA fragments are attached to the surface of flow cell channels by capturing oligonucleotides which are capable of hybridizing to the adapter ends of the fragments. The DNA fragments are then extended and bridge amplified. After multiple cycles of solid-phase amplification followed by denaturation, an array of millions of spatially immobilized nucleic acid clusters or colonies of single-stranded nucleic acids are generated. Each cluster may include approximately hundreds to a thousand copies of single-stranded DNA molecules of the same template. The Illumina platform uses a sequencing-by-synthesis method where sequencing nucleotides comprising detectable labels (e.g., fluorophores) are added successively to a free 3′hydroxyl group. After nucleotide incorporation, a laser light of a wavelength specific for the labeled nucleotides can be used to excite the labels. An image is captured and the identity of the nucleotide base is recorded. These steps can be repeated to sequence the rest of the bases. Sequencing according to this technology is described in, for example, U.S. Patent Publication Application Nos. 2011/0009278, 2007/0014362, 2006/0024681, 2006/0292611, and U.S. Pat. Nos. 7,960,120, 7,835,871, 7,232,656, and 7,115,200, each of which is incorporated herein by reference in its entirety.

According to the present invention a plurality of reads will be obtained. As used herein, the term “read” refers to a sequence read from a portion of a nucleic acid sample. Typically, a read represents a short sequence of contiguous base pairs in the sample. The read may be represented symbolically by the base pair sequence in A, T, C, and G of the sample portion, together with a probabilistic estimate of the correctness of the base (quality score).

According to the present invention, the quality of the generated sequences can be determined, so as to remove low-quality nucleic acid sequences. The high-quality nucleic acid sequences isolated in step b) are preferably sequences with a quality score above a predetermined threshold, preferably a Phred score higher than 20. As used herein, the term “Phred score” has its general meaning in the art and represents the quality of the identification of the nucleobases generated by automated sequencing. The higher the Phred score, the higher the quality. For example, a Phred score of 10 stands for a 90% base call accuracy, and a Phred score of 20 is correlated with a 99% base call accuracy. In addition, an informative score of the nucleic acid sequences can be calculated for additional filtering in order to keep only sequences which contain a meaningful amount of information. For example, a homopolymeric sequence contains little identifying information because it can correspond to many different genomes.

The host cellular nucleic acid sequences can then be subtracted from the obtained high-quality/informativity sequences, so as to obtain only non-animal nucleic acid sequences out of them. Any other means for isolating at least one non-animal high-quality nucleic acid sequence can be implemented.

Subsequent rounds of depletion can advantageously be carried on so as to remove other types of nucleic acid sequences, e.g. mammals, insects, vegetal sequences, etc.; so as to keep only the sequences of interest, e.g. parasites, fungi, bacteria, or viruses. These sequences correspond to the infectious agents to be identified, the sequences of which are then compared to a plurality of known infectious agent sequences so as to identify a closest known sequence out of this plurality of known sequences.

The plurality of known nucleic acid sequences of step d) can for instance consist in a database. Typically, such a database comprises bacterial, viral, fungal and/or parasitic nucleic acid sequences. For instance, such a database may derive from the National Center for Biotechnology Information (NCBI) database. Typically, it comprises an enriched NCBI database, consisting of an NCBI database to which known sequences of interest have been added so as to provide a plurality of known sequences which is as relevant as possible given the origin of the initially provided sample of nucleic acid sequence. NCBI databases advantageously use a taxonomic classification numbering every phylogenetic nod, which allows to identify a taxon even if the taxon has several names, regardless of the name used in the database.

In order to determine the nucleic acid sequence which shares the highest amount of similarities with the sequence to identify, several approaches can be used. A preferred approach consists in iteratively comparing the sequence to be determined to the plurality of known sequences. Different parameters can be taken into account, such as length of common portions, amount of common portions, etc. In addition, non-informative portions of the sequence can be identified by any known mean and given a lower weight in the calculation at any time in the analysis. Known means for calculating phylogenetic distances between nucleic acid sequences can be used to this end as well.

The method according to the present invention can further comprise a step consisting in checking whether the amount of similarities between the closest known sequence and the at least one non-human high-quality nucleic acid sequence is above a predetermined threshold. Without such a step, the method according to the present invention will always return a result corresponding to the closest identified sequence. However, if the sequences are not similar enough, it may be better not to return any result, hence the predetermined threshold to characterize the similarity between the sequence to identify and the output closest sequence.

The threshold needs to be chosen carefully and will depend on the infectious agent to identify. Indeed, even a rather remote sequence can suffice to identify an infectious agent in some cases, whereas a high similarity can be needed in order to reliably identify other infectious agents. For instance, fast mutating viruses would not be assigned the same threshold as fungi.

In some embodiments, the number of sequences correctly identified and their relative amount (ratio) to human sequences are calculated, compared with those of the environmental (negative) control and used to measure the amount of the infectious agent(s) present in the sample for interpretation or its RNA expression, according to experience in the pathogenesis of infectious diseases, so as to report the presence of the infectious agent as compatible with being causative of the infectious disease according to its ratio.

In some embodiments, an interpretation of the positive control can be further used to validate the overall process.

In some embodiments, a specific report containing all control results and numerous indicators of the validity is provided. The method according to the present invention can further comprise a step consisting in generating an analysis report, preferably an analysis report in a format of interest. A format of interest is preferably a format which can be read on most devices such as txt, html or pdf documents.

Typically, the overall process from the sample to the final report is conform to the ISO EN NF 15189 norm (diagnostic for medical laboratories).

The method according to the present invention is very useful to identify bacteria, viruses, fungi and parasite based on their genomic DNA and genomic/expressed RNA; it is particularly efficient at identifying all of these pathogens based exclusively on their genomic (RNA viruses) and/or expressed RNA sequences (including for pathogens the genome of which is a DNA).

The method is of particular interest to identify fungi as known identification methods are not as successful with fungi as they are with other infectious agents.

Therefore, the provided sample of nucleic acid sequences of step a. can advantageously be a sample containing fungus RNA sequences.

Most fungi share an important part of their genomes in common. This part of the genome common to most fungi is thus not informative. It is therefore advisable to use RNA sequences rather than DNA sequences in order to identify a fungus.

There are genes which are highly specific for given fungi and, as such, constitute discriminant fungal genes. Exemplary discriminant fungal genes include: i) nuclear ribosomal RNA gene large subunit (D1-D2 domains of 26/28S); ii) the complete internal transcribed spacer region (ITS1/2); iii) partial β-tubulin II (TUB2); iv) γ-actin (ACT); v) translation elongation factor 1-α (TEF1α) and translation elongation factor 3 (TEF3); vi) the second largest subunit of RNA-polymerase II (partial RPB2, section 5-6); vii) a small ribosomal protein necessary for t-RNA docking; viii) the 60S L10 (L1) RP; ix) DNA topoisomerase I (TOPI); x) phosphoglycerate kinase (PGK); xi) protein LNS2 (as described in Stielow J B et al; Persoonia 2015).

In the present specification, the name of each of the various genes of interest refers to the internationally recognized name of the corresponding gene, as found in internationally recognized gene sequences and protein sequences databases, including in the database from the HUGO Gene Nomenclature Committee that is available notably at the following Internet address: www.gene.ucl.ac.uk/nomenclature/index.html. Through these internationally recognized sequence databases, the nucleic acid and the amino acid sequences corresponding to each of the marker of interest described herein may be retrieved by the one skilled in the art.

According to the present invention, the plurality of known sequences of step d) comprises at least one discriminant fungal gene of interest as defined above.

Typically, the method of the present invention is performed by a computer program that includes several modules as described in EXAMPLE 2. Briefly said, computer program may comprise a first module used to eliminate poor quality sequences (Phred score<20), non-informative homopolymeric sequences, and human sequences using hg19 database. The second module may carry out an identification of the infectious agent using a cleaned database. After this identification step, each infectious agent sequence from each sample (patient/animal samples, environmental control and blank samples) is tagged with identification. The sequences from the patient samples are cleaned using those found in common in the environmental control. A ratio (number of microorganism sequences/human or animal sequences) is then determined for each remaining microorganism at species level for bacteria, viruses and parasites, and at genus level for fungi. All identification that exceeds a certain amount is interpreted as positive. Especially for fungi, the reliability of identification at species level can be checked using a dedicated module. Said module is based on a Simpson index calculated from distribution of species identified from sequences belonging to the same identified genus. When the distribution index is high, this indicates that the sequences all belong to one species, supporting the idea that the information is reliable. In this case, the species is identified. When the index is low, a heatmap of fungal species identification is calculated. This consists in using only the fungal sequences belonging to genes known to be identifying by means of databases of the selected fungal genes (i.e. the discriminant fungal genes). At the end of this step, if at least 3 different identifying genes from the same species are present, the “species” information is validated. Otherwise, only the genus is returned.

Thus, a further object of the present invention is a computer program product comprising code configured to, when executed by a processor or an electronic control unit, perform the method according to the invention.

In some embodiments, the computer program of the present invention is implemented on a computer using well-known computer processors, memory units, storage devices, computer software, and other components. Typically, the computer contains a processor, which controls the overall operation of the computer by executing computer program instructions which define such operation. The computer program instructions may be stored in a storage device (e.g., magnetic disk) and loaded into memory when execution of the computer program instructions is desired. The computer also includes other input/output devices that enable user interaction with the computer (e.g., display, keyboard, mouse, speakers, buttons, etc.). One skilled in the art will recognize that an implementation of an actual computer could contain other components as well.

In some embodiments, the computer program of the present invention is implemented using computers operating in a client-server relationship. Typically, in such a system, the client computers are located remotely from the server computer and interact via a network. The client-server relationship may be defined and controlled by computer programs running on the respective client and server computers. In some embodiments, the results may be displayed on the system for display, such as with LEDs or an LCD. Accordingly, in some embodiments, the algorithm can be implemented in a computing system that includes a back-end component, e.g., a data server, or that includes a middleware component, e.g., an application server, or that includes a front-end component, e.g., a client computer having a graphical user interface or a Web browser through which a user can interact with an implementation, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiments, the computer program of the present invention is implemented within a network-based cloud computing system. In such a network-based cloud computing system, a server or another processor that is connected to a network communicates with one or more client computers via a network. A client computer (e.g. a mobile device, such as a phone, tablet, or laptop computer) may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For instance, the physician may register the parameters (i.e. input data) on, which then transmits the data over a long-range communications link, such as a wide area network (WAN) through the Internet to a server with a data analysis module that will implement the algorithm and finally return the output (e.g. score) to the mobile device. In some embodiments, the output results can be incorporated in a Clinical Decision Support (CDS) system. These output results can be integrated into an Electronic Medical Record (EMR) system.

Another object of the present invention is a kit for detecting, identifying, an infectious agent comprising:
a sample provider configured to be provided with a sample of nucleic acid sequences,
means for implementing the method according to the invention, and
means for displaying results based on a closest known sequence.

The method, kit and computer program of the present invention is particularly suitable for making accurate detection and identification of infectious agents that can be difficult to identify as many samples include flora or background colonization organisms. The method, kit and computer program of the present invention can thus be suitable for classifying, quantifying and/or characterizing an infectious agent. In particular, the method ensures a quick, efficient, and useful identification of infectious agents and thus present many advantages in clinical practice for the diagnosis of infections and in public health surveillance. For example, the method of the present invention may be used where a patient is suspected of suffering from an infectious disease and a clinician may take one or more samples from the patient to determine what infectious agent(s) is/are responsible for said infection. The clinician may indeed desire to know whether the patient has a viral infection, a bacterial infection, a fungal infection, a parasite infection, etc, in order for him/her to look at the results at different levels and provide potential options for treatment when available. In particular, once the infectious agent(s) is (are) identified, additional available clinical and laboratory data can be helpful in determining whether or not the detected infectious agent is pathogenic (i.e. causing disease) in the host organisms (e.g. human or animal patient). The detection of the presence of a potential infectious agent in a clinical sample does not necessarily mean that it is causing disease; the potential pathogen could be a colonizer, for instance, or a bystander and have nothing to do with the host organism's illness. If the identified infectious agent is deemed to be pathogenic by clinical and oilier criteria, the detection can be used to guide clinical interventions, which can include: (1) antimicrobial drug therapy (e.g. prescribing or administering a targeted antimicrobial agent), (2) antimicrobial drug discontinuation (e.g. discontinuing a drug that was administered empirically in the absence of a definitive diagnosis), (3) vaccination, if a vaccine is available and efficacious after infection (e.g. rabies), and (4) medical procedures (e.g. valve replacement in cases of fungal endocarditis, for which antifungal therapy alone is ineffective). The failure to detect an infectious agent may also be clinically useful to exclude the presence of an infection as the cause of illness, which can guide clinicians to treat for noninfectious causes (e.g. administering intravenous immunoglobulin and corticosteroids that would be suitable for the treatment of autoimmune disease, etc). The method, kit and computer program of the present invention may also be used in blood bank testing, food and water quality testing, environmental testing, animal testing, animal health, or any other area that may be assisted by quickly and efficiently identifying the presence of an infectious agent.

The invention will be further illustrated by the following figures and examples. However, these examples and figures should not be interpreted in any way as limiting the scope of the present invention.

### FIGURES

FIG. 1: flow chart of the study of example 1

FIG. 2. (a) Proportions of negative, monomicrobial and polymicrobial samples detected by culture, targeted metagenomics (TM) and shotgun metagenomics (SM) in necrotic samples from the 34 patients with necrotizing soft-tissue infections (NSTIs). (b) Number of microorganisms identified in the 34 patients with NSTIs by culture, TM and SM. GP, Gram positive; GNB, Gram-negative bacilli. (c) Sensitivity of each method for the detection of enterobacteria (including Escherichia coli), nonfermentative (NF) GNB, Gram-positive cocci (GPC), anaerobic bacteria and all microorganisms, based on the combined results of the three methods. (d) Venn diagram showing the number of samples for which each method provided the best possible pathogen identification, based on the combination of results from the three methods.

FIG. 3. (a) Comparison of quantitative shotgun metagenomic (SM) ratios of bacterial-to-human sequences vs. semiquantitative bacterial load estimated by culture (+, ++, +++, ++++). (b) Comparison of bacterial the load calculated from SM ratios in samples collected from healthy and necrotic areas.

### EXAMPLE 1

The results of example 1 have been published in Br J Dermatol. 2020 July; 183(1):105-113 incorporated by reference.

### SUMMARY

Background Necrotizing soft-tissue infections (NSTIs) are life threatening, requiring broad-spectrum antibiotics. Their aetiological diagnosis can be limited by poor performance of cultures and administration of antibiotics before surgery.

Objectives We aimed (i) to compare 16S-targeted metagenomics (TM) and unbiased semiquantitative panmicroorganism DNA- and RNA-based shotgun metagenomics (SM) with cultures, (ii) to identify patients who would best benefit from metagenomics approaches and (iii) to detect the microbial pathogens in surrounding non-necrotic ‘healthy’ tissues by SM-based methods.

Methods A prospective observational study was performed to assess the analytical performance of standard cultures, TM and SM on tissues from 34 patients with NSTIs. Pathogen identification obtained with these three methods was compared.

Results Thirty-four necrotic and 10 healthy tissues were collected from 34 patients. The performance of TM was inferior to that of the other methods (P<0.05), whereas SM performed better than standard culture, although the result was not statistically significant (P=0.08). SM was significantly more sensitive than TM for the detection of all bacteria (P=0.02) and more sensitive than standard culture for the detection of anaerobic bacteria (P<0.01). There was a strong correlation (r=0.71, Spearman correlation coefficient) between the semi-quantitative abundance of bacteria in the culture and the bacteria-to-human sequence ratio in SM. Low amounts of bacterial DNA were found in healthy tissues, suggesting a bacterial continuum between macroscopically ‘healthy’ and necrotic tissue.

Conclusions SM showed a significantly better ability to detect a broader range of pathogens than TM and identify strict anaerobes than standard culture. Patients with diabetes with NSTIs appeared to benefit most from SM. Finally, our results suggest a bacterial continuum between macroscopically ‘healthy’ non-necrotic areas and necrotic tissues.

### Standard Bacteriological Procedures

All biopsies were tested using a standardized bacteriological procedure, according to established guidelines [1]. The biopsies were ground in a sterile disposable tube containing 3 mL isotonic solution and steel beads for 210 s at 50-60 Hz (IKA® Ultra-Turrax® Tube Drive, Staufen, Germany). Part of the ground material (approximately 10-100 mg) was transferred into a Tempus Blood RNA Tube (ThermoFisher Scientific, Waltham, MA, USA) and frozen at −80° C. for metagenomics studies. The remaining part was used to seed the following media: Polyvitex (five days, 5% CO2), colistin nalidixic acid blood plate, trypticase-soy agar and Drigalski plate (48 h, aerobic), blood agar plate (five days, anaerobic), and thioglycolate liquid broth (five days), as recommended by the European Society of Clinical Microbiology and Infectious Diseases (ESCMID)[1].

Bacterial colonies were identified using matrix-assisted laser desorption/ionization time-of-flight mass spectrometry (MALDI-TOF, Beckman-Coulter, Sacramento, CA, USA) and semi-quantitatively counted according to internal charts (+: 1 to 10 unit forming colonies (UFC), ++: 11 to 100 UFC, +++: 101 to 1000 UFC, and ++++: >1001 UFC). Positive blood cultures were managed as recommended by the ESCMID.[1] Antimicrobial susceptibility testing was performed using the disc diffusion method and interpreted according to the 2014 recommendations of the Antibiogram Committee of the French Society for Microbiology [2].

### Metagenomics Procedures

### Extraction and Controls

An unbiased DNA-RNA extraction procedure was applied to all biopsies before performing targeted metagenomics (TM) or shotgun metagenomics (SM). Briefly, pre-extraction by bead homogenisation, combined with chemical cell disruption, was followed by extraction using QiaSymphony (Qiagen, Hilden, Germany).

A negative control was tested in each M or SM run. Positive controls were used to evaluate the performance of the metagenomics techniques for the detection of bacteria, viruses, and fungi. The 10-mL positive-control lot was produced by mixing the following microorganisms: (i) bacteria, including Gram-positive and Gram-negative aerobic and anaerobic species; (ii) viruses, including enveloped and non-enveloped RNA and DNA viruses; and (iii) fungi, including filamentous and non-filamentous pathogens. Aliquots of 500 μL were produced, frozen at −80° C., and used as positive controls.

### Targeted Metagenomics

TM included the study of four amplicon libraries: domains V1-V2 (16S-V1V2) [3] and V3-V4 (16S-V3V4) [4] of the bacterial 16S rRNA gene and the two ribosomal fungal internal transcribed spacer (ITS) regions ITS1 and ITS2 [5]. Each amplicon was prepared from 5 μL extract following the “16S Metagenomic Sequencing Library Preparation protocol” provided by the manufacturer (Illumina, San Diego, CA, USA). For each library, the quality was evaluated using a D1000 ScreenTape on a TapeStation (Agilent, Santa Clara, CA, USA) and the quantity using the Quant-it dsDNA Assay kit (ThermoFischer, Waltham, MA, USA) on a Mithras LB 940 (Berthold Technologies, Bad Wildbad, Germany). All libraries were normalized to 4 nM, pooled, and denatured before pair-end sequencing (v3, 2×300 bp) on a MiSeq device (Illumina, San Diego, CA, USA). The targeted bacterial and fungal regions were sequenced according to the manufacturer's instructions [6] and the sequences compared to those in a dedicated database using our in-house software PyroMIC® [5]. Briefly, after merging pair-end sequences, reads<50 bp and sequences with Phred quality scores<20 were removed. Chimeric sequences were detected by comparing the identifications provided by both sense and anti-sense reads. If identifications were not concordant, the sequences were considered chimeric and removed. The remaining sequences were blasted against the RefSeq database (release 85, November 2017) for 16S rDNA [7] and an in-house fungal database based on the cleansed NCBI database (November 2017) [8]. Bacteria were identified using sequences>300 bp in length with an e-value<10⁻¹⁵⁰ and identity>97% and fungi were identified using sequences>300 bp in length with an e-value<10⁻¹⁸⁰ and identity>99%. Only identifications representing at least 1% of the total number of sequences and a minimal number of 100 attributed sequences were considered.

### Shotgun Metagenomics

SM DNA libraries were prepared using 5 μL extract at 0.2 ng/μL and Nextera XT DNA (Illumina, San Diego, CA, USA), according to the manufacturer's protocol¹⁶. RNA libraries were prepared in parallel, as already reported¹⁷, using 10 μL extract at 10 ng/μL and the Human RiboZero TruSeq Stranded Total RNA Library Prep Kit (Illumina, San Diego, CA, USA). The quality and quantity of each library were assessed using the same protocol as for TM. The DNA and RNA libraries were tagged to ensure separate analysis of DNA and RNA. DNA and RNA were then normalized to equal concentrations (1.8 pM) before pooling, denaturation, and pair-end sequencing using the High Output Kit v2, 2×150 bp on a NextSeq500 Illumina device (Illumina, San Diego, CA, USA) [9].

After sequencing, non-human RNA and DNA were analysed separately using our in-house MetaMIC® software (IDDN.FR.001.160012.000.S.C.2018.000.31230), composed of a mosaic of modules. The pair-end sequences, composed of R1 and R2 files, were analysed using the identification of R1 first. If R1 was identified with a reference, R2 was tested for identification in a window of 1000 bp around the position of identification of R1 in the reference. Only identified R1/R2 couples were retained for the final count of identified microorganisms. Sequences with Phred scores<20 were removed. Human sequences were removed using the hg19 database (Full data set GRCh37/hg19, feb 2009). The identification of non-human sequences and genome reconstruction were performed using various databases, including a cleaned NCBI nt and nr (Genbank release 215, October 2016) database, which contains all known microbes, and a specific in-house bacterial, fungal, and viral database. For each identified species, the negative control sequences were subtracted from those of the samples after normalization of the number of corresponding sequences to the total number of sequences. If there were more than 100 identifying sequences, the corresponding species was considered to be present in the sample and the sample positive. Relative quantification was performed for bacteria using the bacterial sequence/human sequence ratio.
1. Cornaglia G, Courcol R, Herrmann J. European manual of clinical microbiology. 2010:215-22.
2. Bonnet R, Bru J, Caron F, et al. Comité de l'antiobiogramme de la Société Française de Microbiologie, Recommandations 2014. Available at: www.sfm-microbiologie.org/UserFiles/files/casfm/CASFM_EUCAST_V1_0_2014 (1).pdf.
3. Kuczynski J, Lauber C L, Walters W A, et al. Experimental and analytical tools for studying the human microbiome. Nature reviews Genetics 2011; 13(1): 47-58.
4. Klindworth A, Pruesse E, Schweer T, et al. Evaluation of general 16S ribosomal RNA gene PCR primers for classical and next-generation sequencing-based diversity studies. Nucleic acids research 2013; 41(1): el.
5. Sitterle E, Rodriguez C, Mounier R, et al. Contribution of Ultra Deep Sequencing in the Clinical Diagnosis of a New Fungal Pathogen Species: Basidiobolus meristosporus. Frontiers in microbiology 2017; 8: 334.
6. Illumina. 16S Metagenomic Sequencing Library Preparation. Available at: https://web.uri.edu/g sc/files/16 s-metagenomic-library-prep-guide-15044223-b.pdf.
7. Tatusova T, Ciufo S, Fedorov B, O'Neill K, Tolstoy I. RefSeq microbial genomes database: new representation and annotation strategy. Nucleic acids research 2015; 43(7): 3872.
8. Pruitt K D, Tatusova T, Maglott D R. NCBI reference sequences (RefSeq): a curated non-redundant sequence database of genomes, transcripts and proteins. Nucleic acids research 2007; 35(Database issue): D61-5.
9. Illumina. Available at: support.illumina.com/content/dam/illumina-support/documents/documentation/chemistry_documentation/sampleprep s_nextera/nextera-xt/nextera-xt-library-prep-reference-guide-15031942-03.pdf.

### Study Population

All adult patients hospitalized during the study period for clinical suspicion of NSTI were included (FIG. 1). Confirmation of the diagnosis was based on surgical findings with a necrotizing component involving any or all of the layers of the soft-tissue compartment, from dermis and subcutaneous tissue to deeper fascia and muscle.1 Samples were available in sufficient amounts for metagenomics studies for 34 of the 66 patients with a suspected NSTI. These 34 patients comprise the study group. The following parameters were recorded in standardized files: age, sex, comorbidities, intensive care unit admission, direct admission or transfer from another facility, delay of surgery after admission, antibiotics administrated before surgery and clinical outcome (including death). Antibiotics were adapted according to the results of the standard culture-based approach, which was considered the standard of care (see below).

### Samples

The patients underwent extensive debridement of all necrotic and nonvascularized tissues, including skin, subcutaneous fat, fasciae and muscles. When necessary, deep fasciotomies were performed. Amputated limbs were not sampled. Deep biopsies were collected aseptically during the surgical procedures, both from the interface between safe and necrotic tissues (‘infected samples’) and, for a subgroup of 10 of the 34 patients, from non-necrotic tissues, defined as macroscopically healthy tissue surrounding the focus of infection (‘healthy samples’). The samples were sent within 2 h at room temperature to the department of microbiology (open 24 hours per day, 7 days per week).

### Standard Microbiological and Metagenomics Procedures

All biopsies were tested by (i) a standardized microbiological procedure, (ii) TM of the V1-V2 (16S-V1V2) and V3-V4 (16S-V3V4) domains of the bacterial 16S ribosomal gene and the two ribosomal fungal internal transcribed spacer (ITS) regions ITS1 and ITS2, and (iii) an unbiased in-house semi-quantitative panmicroorganism DNA- and RNA-based SM method, MetaMIC. Our SM method was considered to be unbiased because no DNase or capture enrichment was used during the extraction step. All identified microorganisms were considered to be responsible for the infectious process.

### Statistical Analyses

The three diagnostic methods were compared for their ability to identify the bacterial aetiologies of the NSTIs. In the absence of a gold standard, sensitivity was evaluated by comparing the results provided by one single method with those obtained by the sum of the information generated by the three methods (culture, TM and SM). The relationship between the three methods was assessed using the kappa coefficient, the strength of which was considered slight between and 0.20, fair between 0.21 and 0.40, moderate between 0.41 and 0.60, substantial between 0.61 and 0.80, and almost perfect between 0.81 and 1.00.

Unadjusted comparisons based on the t-test or Mann-Whitney test for quantitative data and the v2-test or Fisher's exact test for categorical data were performed to understand which patient characteristics were associated with the positive contribution of TM and/or SM.

Data are described as the mean±SD or median (interquartile range) for continuous data, depending on distribution normality, and as proportions (%) for categorical data. Two-tailed P-values<0.05 were considered significant. Statistical analyses were performed using Stata software version 14•1 (StataCorp LP, College Station, TX, U.S.A.).

### Results

### Patients

Sixty-six patients were eligible. Samples were available in sufficient amounts for metagenomics studies for 34, who were included in the study (FIG. 1). Thirty-four necrotic samples and 10 ‘healthy’ samples were collected from the patients. Seventy-four percent of patients (25 of 34) presented with comorbidities, the most frequent of which were diabetes mellitus (38%, 13 of 34), immunosuppression (29%, 10 of 34) and obesity (26%, nine of 34). Previous exposure to antibiotics was reported for 68% of patients (23 of 34). All patients underwent at least one debridement surgery, and 97% (33 of 34) were empirically treated with a broad-spectrum antibiotic, according to local guidelines. Fifty percent of patients (17 of 34) were admitted to the intensive care unit and 6% (two of 34) died during hospitalization.

### Assessment of the Diagnostic Value of Targeted and Shotgun Metagenomics Relative to Standard Cultures

### Results of Standard Culture

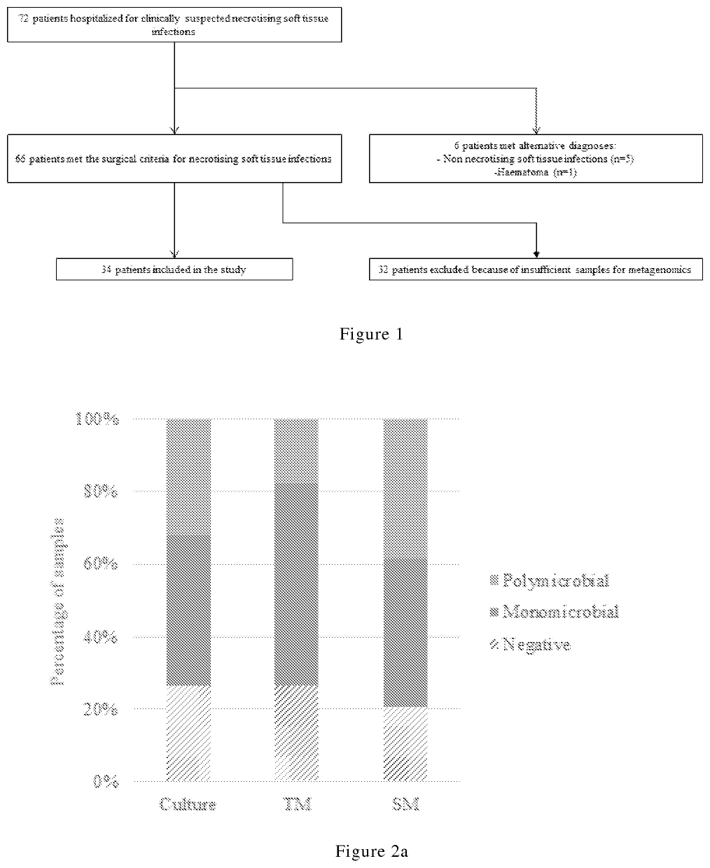

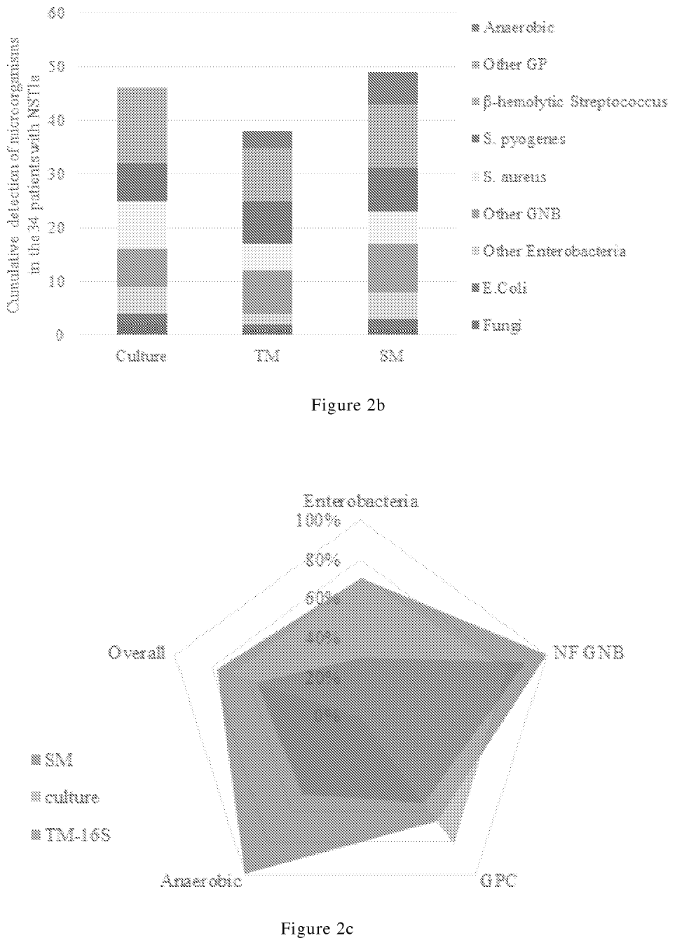

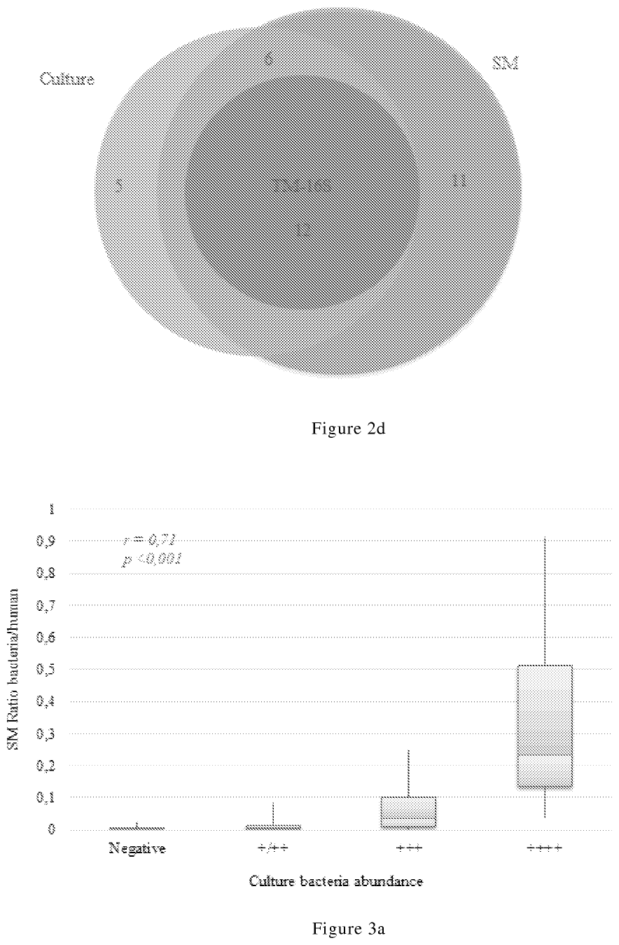

Infected samples were positive for 74% of the patients (25 of 34) by classical culture methods (FIG. 2a). The cultures identified only one bacterial species in 41% of cases (14 of 34): Staphylococcus aureus (five cases), Streptococcus pyogenes (four cases), Pseudomonas aeruginosa (three cases), Haemophilus influenzae (one case) and coagulase-negative staphylococci (one case) (FIG. 2b). Polymicrobial cultures were found in 32% of cases (11 of 34): S. aureus (four cases), S. pyogenes (three cases), Enterobacteria (nine cases), nonfermentative Gram-negative bacilli (NF-GNB) (three cases), enterococci (four cases), others (three cases) and a mix of Candida albicans and Candida tropicalis (one case) (FIG. 2b). No anaerobic bacteria were found.

### Results of the Metagenomics Methods

TM gave positive results (presence of bacteria and/or fungi) for 44% (15 of 34) of necrotic tissues using 16S V1-V2 (mean 74 890±34 158 sequences per sample) and for 74% (25 of 34) using 16S V3-V4 (mean 282 681±85 776 sequences per sample) (FIG. 2a). There was no discrepancy for the bacterial identification between the two 16S targets, and because of an apparent lack of sensitivity of V1-V2, only the V3-V4 results were used for comparison of the technologies. SM gave positive results for 79% (27 of 34) of the necrotic samples using DNA and RNA (mean 35 468 679±11 964 012 RNA sequences per sample and 39 218 559±4 969 662 DNA sequences per sample). The quality of the sequences (Q30) was above that recommended by the manufacturer (>90%). All pathogens in the positive controls were adequately identified (data not shown). Sequences are available in the National Center for Biotechnology Information database (PRJNA553328).

Monomicrobial infection was reported in 53% of cases (18 of 34) by TM: S. aureus (two cases), S. pyogenes (seven cases), Streptococcus dysgalactiae (one case), Escherichia coli (one case), NF-GNB (five cases), Clostridium perfringens (one case) and others (one case). Monomicrobial infection was reported in 38% of cases (13 of 34) by SM: S. aureus (three cases), S. pyogenes (four cases), E. coli (one case), NF-GNB (three cases) and others (two cases) (FIG. 2b). Multiple bacterial species were identified in 21% of cases (seven of 34) by TM: S. aureus (three cases), S. pyogenes (one case), Streptococcus agalactiae (one case), Enterobacteria (two cases), NF-GNB (two cases) and C. albicans (one case) (FIG. 2b), whereas SM showed polymicrobial infection in 41% (14 of 34) of cases: S. aureus (three cases), S. pyogenes (four cases), Enterobacteria (seven cases), NF-GNB (four cases), anaerobic bacteria (seven cases), C. albicans (one case) and others (four cases). No viral DNA or RNA was identified in any of the 34 patients.

### Comparison of the Three Methods for Microbial Identification in Necrotic Samples

Positive results, defined as the presence of at least one microbial species, were obtained for 74% (25 of 34) of the samples by culture, 74% (25 of 34) by TM and 79% (27 of 34) by SM.

Overall, the sensitivities for the detection of Gram-positive cocci, Enterobacteria, NF-GNB and anaerobic bacteria were 81%, 70%, 70% and 0% by culture; 56%, 30%, 80% and 50% by TM; and 67%, 70%, 90% and 100% by SM, respectively. SM was significantly more sensitive than TM for the detection of all bacteria (P=0.02) and more sensitive than standard culture for the detection of anaerobic bacteria (P<0.01). Standard culture was more sensitive than TM for the detection of Gram-positive cocci (P=0.04) (FIG. 2c).

Only 15% of samples (five of 34) yielded a negative result in the three tested methods. The three methods identified the full spectrum of microbes in 21% of cases (seven of 34), whereas all microorganisms were identified by both culture and SM in 18% (six of 34) of additional cases. In the remaining 16 cases, complete identification was obtained by culture in five cases and by SM in 11 cases (FIG. 2d). We found TM to be inferior to the two other methods to provide complete identification of infectious agents in NSTIs (P=0.02), whereas the number of correct identifications by SM was higher than that obtained by culture, although the result was not statistically significant (P=0.08).

### Relationship Between the Three Methods

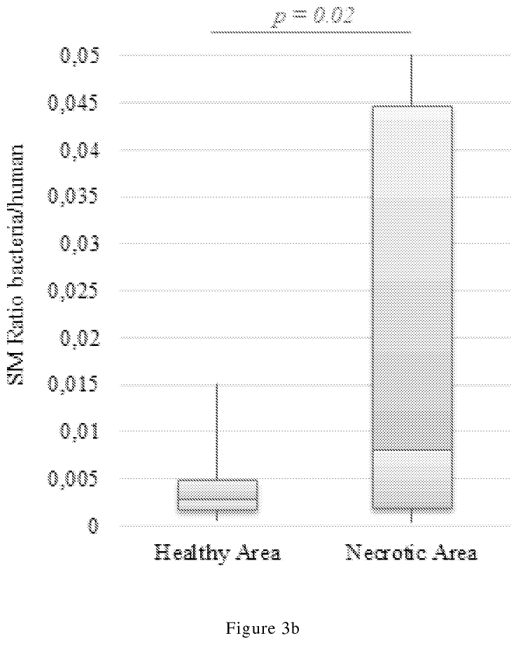

In summary, the kappa coefficient was 0.22 (50% agreement, P=0.03) between culture and TM, 0.41 (61% agreement, P<0.001) between culture and SM, and 0.47 (65% agreement, P<0.001) between TM and SM. There was a strong correlation between bacterial semiquantitation in culture and the bacteria-to-human sequence ratio in SM (r=0.71, P<0.001; FIG. 3a).

Analysis of Patients in Whom Shotgun Metagenomics Yielded More Complete Pathogen Identification than the Other Methods

SM yielded more complete pathogen identification than the two other methods for 11 patients. A comparison of these patients with the 23 others showed diabetes mellitus to be the only significant differentiator (odds ratio 5.0, 95% confidence interval 1.1-23.2; P=0.04). We also observed a higher ratio for patients over 75 years of age, although the result was not statistically significant (odds ratio 4.0, 95% confidence interval 0.8-16.7; P=0.08). None of the other tested characteristics (including sex, first admission to the intensive care unit, obesity, immunosuppression, preadmission antimicrobial therapy, previous steroid intake, previous surgical procedure, number of days of hospitalization or NSTI type) was associated with an improved diagnosis using SM.

### Assessment of Non-Necrotic ‘Healthy’ Tissues

Six of the 10 healthy samples (samples taken from non-necrotic tissues from patients with NSTI) were negative in culture. We observed monomicrobial growth in two cases— S. aureus (one case) and P. aeruginosa (one case)—whereas polymicrobial growth occurred in the two others—E. coli, Enterococcus faecium and S. aureus (one case) and Providencia stuartii, Citrobacter freundii and coagulase-negative staphylococci (one case). Three of these four cases showed complete concordance between healthy and necrotic tissues.

Six healthy samples were negative and four were positive by TM, with a monomicrobial result: S. aureus (one case), S. pyogenes (one case), P. stuartii (one case) and P. aeruginosa (one case). Three of these four cases showed complete concordance between healthy and infected samples. In the nonconcordant case, the pathogen was found in the healthy but not the necrotic tissue.

Only three of 10 ‘healthy’ non-necrotic samples were negative by SM. Five samples were monomicrobial—S. aureus (two cases), S. pyogenes (one case) and P. aeruginosa (two cases)—and two were polymicrobial: P. stuartii, C. freundii and Morganella morganii (one case), and E. coli, E. faecium and S. aureus (one case). Six of these seven cases showed complete concordance between ‘healthy’ and necrotic samples. The quantitative ratio of bacterial-to-human sequences was significantly smaller in healthy’ than in necrotic tissues for the 10 patients tested in both areas (P=0.02, FIG. 3b).

### Conclusion

We aimed to assess the performance of an original, unbiased, semiquantitative, panmicroorganism DNA- and RNA-based SM method on prospectively collected tissues from patients with NSTIs.

Two different metagenomics approaches, TM and unbiased SM, were used in parallel, along with standard culture, to assess patients with NSTIs. Overall, SM was significantly better than TM at detecting a broad range of pathogens, and significantly better than culture at identifying strict anaerobes. TM and SM identified strict anaerobes significantly better than standard culture and enabled the identification of more NF-GNB.

In conclusion, SM is a new NGS-based method that is well adapted to pathogen identification through the detection of a wide variety of microbes in cutaneous tissues. Although it is still complex to set up for routine use, the results of SM correlate with those of classical culture-based approaches, with better sensitivity for polymicrobial and anaerobic infections. Strategies using SM-based diagnosis may change the landscape of infectious diseases by enabling treating physicians to make personally tailored decisions based on complete microbiological profiling of their patients.

### EXAMPLE 2

### Summary

Deep cutaneous mycoses in transplant recipients are infections frequently caused by fungal invasion of the skin and subcutaneous tissue, frequently arising after traumatic inoculation. They often involve rare or emerging opportunistic fungal pathogens originating from the soil, yielding species identification difficult. Shotgun Metagenomics (SMg) is a comprehensive method for pan-pathogen detection, particularly accurate identification of fungi from clinical samples. However, fungal infections have been poorly explored by these techniques thus far, because of an incomplete genetic knowledge and the low value of genome informativeness for its identification. We propose in this study to validate the SMg approach in a cohort of kidney transplant recipients presenting with subcutaneous fungal infection.

Biopsies from 13 kidney transplant patients with fungal subcutaneous infection, as characterized by conventional mycology techniques (microscopy, culture, mass spectrometry, molecular biology), were tested by SMg. An ISO 15189 accredited pan-pathogen SMg technique routinely used in our laboratory was run after specific pan-pathogen extraction, DNA/RNA library preparation, followed by sequencing with NextSeq500 (Illumina) and analysis with MetaMIC software. An algorithm including informative fungal genes was developed to allow for accurate species identification.

Based on DNA sequences, only 7/13 patients could be diagnosed as positive, while 13/13 patients were screened with a correct identification at the genus level when using RNA sequences. The etiological agents included dematiaceous molds (n=6), hyphomycetes (n=3), dermatophyte (n=2), and mucorales (n=2). Among these 13 patients, 9 had their fungi identified at the species level with high confidence. Fungal loads could be measured with a median 1.93-log higher for RNA load as compared to DNA loads, explaining the difference in sensitivity between the two markers.

In conclusion, metagenomics using unbiased RNA sequencing improves the efficiency of the SMg method to identify fungal pathogens, even from cutaneous biopsies, a difficult matrix because of the low amount of fungal genetic materials it contains versus human genetic materials. We were able to show that under extreme conditions, SMg has the ability to yield reliable fungal identification, confirming its pan-pathogenic spectrum. Moreover, this ISO 15189-certified method proved to be perfectly suited to complex cases of infection involving rare pathogens.

Methods:

Extraction. As described previously [Rodriguez et al; BJD 2019; Deschamps et al; BJD 2019], the biopsies (10-50 mg) were ground in a sterile disposable tube containing 400 μL of isotonic sterile solution and steel beads for 210 s at 50-60 Hz (IKA® Ultra-Turrax® Tube Drive, Staufen, Germany) and transferred into 2 mL Sarsted tubes. A pre-extraction step using bead beating combined with chemical cell disruption solution and Proteinase K was performed, followed by extraction using QiaSymphony (Qiagen, Hilden, Germany). An environmental control (isotonic sterile solution) and positive control (ZymoBIOMICS™ Microbial Community Standards, Ozyme) were tested in each targeted or shotgun metagenomics run. In addition, 5 blank samples (isotonic sterile solution) were used in a separate run to calculate blank of detection (BoD) and limit of detection (LoD) specifically for fungi.

Targeted metagenomics. Targeted metagenomics included the study of two amplicon libraries of the two ribosomal fungal internal transcribed spacer (ITS) regions ITS1 and ITS2 (Sitterle et all, front 2017). Each amplicon was prepared from 5 μL of extract following the “16S Metagenomic Sequencing Library Preparation protocol” provided by the manufacturer (Illumina, San Diego, CA, USA). For each library, the quality and quantity were evaluated by means of D1000 ScreenTape on TapeStation (Agilent, Santa Clara, CA, USA) and Quant-it dsDNA Assay kit (ThermoFischer, Waltham, MA, USA) on Mithras LB 940 (Berthold Technologies, Bad Wildbad, Germany), respectively. All libraries were normalized at 4 nM, pooled and denatured before pair-end sequencing (v3, 2×300 bp) on a MiSeq device (Illumina, San Diego, CA, USA). The targeted bacterial and fungal regions were sequenced according to the manufacturer's instructions [2] and compared to a dedicated database by means of our in-house software PyroMIC® (Sitterle et al. 2017). Briefly, after merging pair-end sequences, reads smaller than 50 bp and sequences with Phred quality score lower than 20 were removed. Chimeric sequences were detected by comparing the identifications provided by both sense and anti-sense reads. When identifications were not concordant, sequences were considered chimeric and removed. The remaining sequences were blasted with in-house fungal database based on the cleansed NCBI database (November 2017) (Pruitt K D; NAR, 2007). The parameters used for proper identification were sequence length greater than 300 bp, an e-value<10-180 and identity>99%. Only identification representing at least 1% of the total number of sequences and a minimal number of sequences>100 attributed sequences were considered.

Shotgun metagenomics experiments. According to the manufacturer's protocol, 5 μL of extract at 0.2 ng/μL were used to prepare DNA shotgun metagenomics libraries by means of Nextera XT DNA (Illumina, San Diego, CA, USA). RNA libraries were prepared in parallel, as already reported [17], using 10 μL of extract at 10 ng/μL and RNA Human RiboZero TruSeq Stranded Total RNA Library Prep Kit (Illumina, San Diego, CA, USA). For each library, the quality and quantity were assessed following the same protocol as with targeted metagenomics. The DNA and RNA libraries were tagged in order to ensure separate analysis of DNA and RNA. DNA and RNA were then normalized at equal concentrations (1.8 pM) before pooling, denaturation and pair-end sequencing by means of High Output Kit v2, 2×150 bp on NextSeq500 Illumina device (Illumina, San Diego, CA, USA) [4].

Shotgun metagenomics data analysis. After sequencing, the generated RNA and DNA sequences were analysed separately with our in-house MetaMIC software, composed of a mosaic of modules. The first module eliminates poor quality sequences (Phred score<20), non-informative homopolymeric sequences, and human sequences using hg19 database (Full data set GRCh37/hg19, feb 2009). The second module carries out the identification of microorganisms using NCBI nt and nr (Genbank release 230, February 2019) cleaned database. After this identification step, each microorganism sequence from each sample (patient samples, environmental control and blank samples) were tagged with identification.

The five blank samples were used to evaluate Mean Blank, Limit of Blank [LoB=Mean Blank+1.65*Stdev Blank] and Limit of detection ratio [LoD=Mean Blank+3.3*Stdev Blank] from false-positive microorganism sequences and LoD was used as positive cut-off for patient samples. [Little, “Method Validation Essentials, Limit of Blank, Limit of Detection, and Limit of Quantitation,” BioPharm International 28 (4) 2015].

The sequences from the patient samples were cleaned using those found in common in the environmental control. A ratio (number of microorganism sequences/human sequences) was then determined for each remaining microorganism at the species level for bacteria, viruses and parasites, and at the genus level for fungi. All identifications that exceeded the LoD were interpreted as positive.

Especially for fungi, the reliability of identification at the species level was checked using a dedicated module. The latter is based on a Simpson index calculated from the distribution of species identification of sequences belonging to a specific genus. When the distribution index was high, the sequences all belonged to one species, supporting the fact that the information was reliable. When the index was low, a heatmap of fungal species identification was calculated.

The heatmap consisted in using only the fungal sequences belonging to genes known to be identifying by means of databases of selected fungal genes (ITS, LSU . . . ). At the end of this step, if at least 3 different identifying genes from the same species had been positive, the “species” information was validated. Otherwise, only the genus was returned.

Results:

Patient biopsies were sequenced by SMg with a median of 40,276,258 [range: 24,919,804-72,8725,50] DNA sequences and 25,049,534 [range: 13,479,338-476,727,578] RNA sequences per sample with a Q30 Quality score>75%, as recommended.

The fungal LoB and LoD ratios based on blank samples were established at 1.00e-6. LoD was used as the lower limit for positivity for patient samples. The DNA and RNA fungal ratios (number of fungal sequences/human sequences) were also calculated and reported. Using the LoD cut-off, 13/13 patients were positive for fungal infection using RNA information, whereas only 6/13 were positive using DNA information. Four additional patient samples harbored DNA sequences from the detected fungi, but they were below LoB, i.e. not in a higher amount than the background noise. The comparison of the DNA and ratios showed a difference of 1.93 log, suggesting that the RNA amounts are approximately 100 times higher than the DNA amounts. Thus, RNA was preferably used to detect the presence or absence of fungi.

The final identification of fungi in patients 3, 4, 6, 8, 10, and 11 was supported by a high Simpson index and the result was delivered without any additional analysis. In contrast, patients 1, 2, 5, 7, 9, 12 were tested by means of Heatmap approaches. Using this additional tool, patients 1, 2 and 12 did not reach the positivity threshold for at least 3 genes and were thus considered positive for Alternaria sp., whereas patients 5, 7 and 9 were identified at the species level, found infected with Alternaria infectoria, Alternaria rosae and Scedosporium apiospermum, respectively.

Comparison of Fungi Identification Approaches

Few differences were seen between the different technologies used. ITS yielded a different result for Scedosporium apiospermum, identified under its sexuate state Pseudallescheria boydii, whereas SMg yielded all identifications at at the genus level. Indeed, Mucor circinelloides is considered as a synonym of Rhizomucor variabilis [Mucormycosis Caused by Unusual Mucormycetes, Non-Rhizopus, -Mucor, and -Lichtheimia Species; Marisa Z. R. Gomes; Clin Microbiol Rev. 2011 April; 24(2): 411-445], and Paecilomyces lilacinus as a synonym of Purpurocillium lilacinum. Nevertheless, SMg was capable to identify fungi at the species level with high confidence in 4 patients for which only the genus level had been identified with other techniques, in addition to viruses and bacteria that were identified in the same analysis.

Discussion:

Shotgun metagenomics is a promising technique that has been poorly evaluated thus far for the diagnosis of fungal infections, especially in the context of atypical fungi in skin biopsies. We report here the evaluation of a pan-pathogenic SMg technique versus usual techniques of fungal diagnosis by culture and by molecular biology using ITS.

Our SMg technique makes it possible to evaluate the background noise to set a reliable detection limit. This method of calculation, added to the use of RNA sequences instead of DNA sequences, made it possible to maximize the sensitivity of the technique to obtain, at the end, a score of 13/13 samples identified as positive with the correct fungus identification. Fungal RNA has been neglected in the past, but its use has 2 advantages, the first relates to the amount of this nucleic acid that is 100 times higher than that of fungal DNA; the second is the specificity of the analysis, as indicated by the absence of identification error in our study (although more patients will need to be tested to confirm this point).

The technique presents an undeniable advantage compared to the other techniques, because it made it possible to ensure correct identification of fungi by selecting the contributive genes of interest. In the present study, the advantage was not obvious because all of the fungi could also be identified by ITS, but they were selected in part on the basis of these previous results. Nevertheless, it is known that ITS regions are not always capable to provide identification at the species level and, moreover, the sensitivity of amplification in this region is closely dependent on the number of fungal nucleic acid copies present in the sample. These two important limitations have no effect on the results of SMg because any gene can be contributive and the number of ITS copies has no effect as the full genome and meta-transcriptome are sequenced. The software's ability to assess the reliability of the information is also an important advantage because, unlike techniques that do not have sufficient species discrimination capacity, SMg results cannot be over-interpreted. For the fungi described in this study, species information had no impact on treatment management because the treatments were identical for all members of the same genus.

When a patient has an infection, it is often difficult to predict that this infection has a fungal origin. This implies that the number of microbiological investigations required to find the cause is very large and requires large sample volumes. However, in the context of deep infection requiring a biopsy, like in our study, the available volume of the biopsy requires that choices are made to carry out conventional approaches. SMg has the advantage of requiring a reasonable sample volume to carry out all the necessary explorations without a priori. Previous studies have demonstrated the capacity of the technique to detect and identify bacteria and viruses, including new, yet unknown pathogens. The present study completes these findings and demonstrates the pan-pathogen detection power of SMg.

### Conclusion

In conclusion, SMg has demonstrated its ability to detect and characterize atypical fungal infections in a complex matrix, alongside other microorganisms, with a sensitivity identical to that of other techniques routinely used in clinical microbiology. This approach without a priori is particularly interesting when the material is in small quantity and the suspected infection is difficult to detect and document by means of conventional techniques.

### REFERENCES

Carrasco-Zuber J E, Navarrete-Dechent C, Bonifaz A, Fich F, Vial-Letelier V, et al. (2016) Cutaneous involvement in the deep mycoses: A review. Part II—Systemic mycoses. Actas Dermosifiliogr 107: 816-822.
Carrasco-Zuber J E, Navarrete-Dechent C, Bonifaz A, Fich F, Vial-Letelier V, et al. (2016) Cutaneous Involvement in the Deep Mycoses: A Literature Review. Part I-Subcutaneous Mycoses. Actas Dermosifiliogr 107: 806-815.
Chiu C Y, Coffey L L, Murkey J, Symmes K, Sample H A, et al. (2017) Diagnosis of Fatal Human Case of St. Louis Encephalitis Virus Infection by Metagenomic Sequencing, California, 2016. Emerg Infect Dis 23: 1964-1968.
Chiu C Y, Miller S A (2019) Clinical metagenomics. Nat Rev Genet 20: 341-355.
Fishman J A (2007) Infection in solid-organ transplant recipients. N Engl J Med 357: 2601-2614.
Fishman J A (2017) Infection in Organ Transplantation. Am J Transplant 17: 856-879.
Gu W, Miller S, Chiu C Y (2019) Clinical Metagenomic Next-Generation Sequencing for Pathogen Detection. Annu Rev Pathol 14: 319-338.
Guegan S, Lanternier F, Rouzaud C, Dupin N, Lortholary O (2016) Fungal skin and soft tissue infections. Curr Opin Infect Dis 29: 124-130.
Illumina. 16S Metagenomic Sequencing Library Preparation. Available at: web.uri.edu/gsc/files/16s-metagenomic-library-prep-guide-15044223-b.pdf.
Nilsson R H, Larsson K H, Taylor A F S, et al. The UNITE database for molecular identification of fungi: handling dark taxa and parallel taxonomic classifications. Nucleic acids research 2019; 47(D1): D259-D64.
Pruitt K D, Tatusova T, Maglott D R. NCBI reference sequences (RefSeq): a curated non-redundant sequence database of genomes, transcripts and proteins. Nucleic acids research 2007; 35(Database issue): D61-5.
Romero F A, Deziel P J, Razonable R R (2011) Majocchi's granuloma in solid organ transplant recipients. Transpl Infect Dis 13: 424-432.
Sitterle E, Rodriguez C, Mounier R, et al. Contribution of Ultra Deep Sequencing in the Clinical Diagnosis of a New Fungal Pathogen Species: Basidiobolus meristosporus. Frontiers in microbiology 2017; 8: 334.

1. A method for identifying an infectious agent comprising:
providing a sample of nucleic acid sequences;
isolating high-quality nucleic acid sequences out of the sample of nucleic acid sequences;
isolating at least one non-animal high-quality nucleic acid sequence out of the high-quality nucleic acid sequences; and
identifying a closest known sequence out of a plurality of known sequences, wherein the closest known sequence shares the highest amount of information with the at least one non-animal high-quality nucleic acid sequence among the plurality of known sequences, and wherein the plurality of known sequences comprises sequences of infectious agents.

2. The method of claim 1 wherein providing a sample of nucleic acid sequences comprises extracting the nucleic acid sequences, and wherein the method further comprises monitoring extracting the nucleic acid sequences so as to generate information comprising at least one of the progress of the extraction and the origin of the sample.

3. The method of claim 1, wherein providing a sample of nucleic acid sequences comprises providing a sample of RNA sequences.

4. The method claim 1, wherein isolating high-quality nucleic acid sequences out of the sample of nucleic acid sequences comprises isolating sequences the quality of which is above a predetermined threshold out of the sample of nucleic acid sequences.

5. The method claim 1, wherein the plurality of known sequences is a database.

6. The method of claim 5, wherein the database comprises an NCBI database.

7. The method claim 1 further comprising checking whether the amount of shared information between the closest known sequence and the at least one non-human high quality nucleic acid sequence is above a predetermined threshold.

8. The method further comprising generating an analysis report.

9. The method claim 1, wherein providing a sample of nucleic acid sequences comprises providing a sample of bacterial, fungal, parasite or viral RNA sequences.

10. The method claim 1 further comprising isolating high-quality nucleic acid sequences out of a sample deprived of any nucleic acid sequences of interest.

11. A kit for identifying an infectious agent comprising:
a sample provider configured to be provided with a sample of nucleic acid sequences,
means for implementing a method for identifying an infectious agent, and
means for displaying results based on a closest known sequence;
wherein the method for identifying an infectious agent comprises:
providing a sample of nucleic acid sequences;
isolating high-quality nucleic acid sequences out of the sample of nucleic acid sequences;
isolating at least one non-animal high-quality nucleic acid sequence out of the high-quality nucleic acid sequences; and
identifying a closest known sequence out of a plurality of known sequences, wherein the closest known sequence shares the highest amount of information with the at least one non-animal high-quality nucleic acid sequence among the plurality of known sequences, and wherein the plurality of known sequences comprises sequences of infectious agents.

12. A computer program product comprising code configured to, when executed by a processor or an electronic control unit, performs
providing a sample of nucleic acid sequences;
isolating high-quality nucleic acid sequences out of the sample of nucleic acid sequences;
isolating at least one non-animal high-quality nucleic acid sequence out of the high-quality nucleic acid sequences; and
identifying a closest known sequence out of a plurality of known sequences, wherein the closest known sequence shares the highest amount of information with the at least one non-animal high-quality nucleic acid sequence among the plurality of known sequences, and wherein the plurality of known sequences comprises sequences of infectious agents.

13. The method of claim 1, wherein the plurality of known sequences comprises sequences of at least one fungal discriminant gene of interest and wherein said identification indicates the infectious agent.

14. The method of claim 6, wherein the NCBI database is an enriched NCBI database.

15. The method of claim 8, wherein the analysis report is in a format of interest.
